(19) United States
(12) Patent Application Publication
(10) Pub. No.: US 2017/0319467 A1
(43) Pub. Date: Nov. 9, 2017

(54) TOPICAL COMPOSITION AND METHOD OF PREPARATION

(71) Applicant: NAOLYS SARL, Cestas Pierroton (FR)

(72) Inventor: Rachid Ennamany, Bordeaux (FR)

(21) Appl. No.: 15/661,938

(22) Filed: Jul. 27, 2017

Related U.S. Application Data
(63) Continuation-in-part of application No. PCT/IB2016/000058, filed on Jan. 28, 2016.

(30) Foreign Application Priority Data
Jan. 30, 2015 (EP) 15 000 285.5

(51) Int. Cl. A61K 8/9789; A61K 8/02; A61K 8/04; C12N 5/04; A61K 8/06; A61Q 19/00; A61Q 17/00; A61Q 5/02; A61Q 1/12; A61K 8/9794; A61Q 1/06; A61Q 1/10; A61Q 19/08

(52) U.S. Cl. CPC A61K 8/9789; A61Q 1/10; A61K 8/022; A61K 8/04; A61K 8/042; C12N 5/04; A61Q 19/08; A61Q 19/00; A61Q 17/005; A61Q 5/02; A61Q 1/12; A61K 8/9794; A61Q 1/06; A61K 8/06; A61K 2800/10; A61K 2800/522; C12N 2500/02; C12N 2523/00

(57) ABSTRACT

A process for the preparation of dedifferentiated and elicited plant cells suitable for topical cosmetic composition, in which dedifferentiated plant cells are elicited following a cycle comprising at least 10 successive darkness period of 20 to 180 minutes separated the one from the other by a lighting period of 1 to 6 hours, under an atmosphere comprising from 1 to 10% by volume CO₂.

### CROSS-REFERENCE TO RELATED APPLICATIONS

The present application is a Continuation-in-part application of International patent application PCT/IB 2016/000058 filed on Jan. 28, 2016 and published under number WO 2016/120713, which claims the priority benefit of European patent application 15 000 285.5 filed Jan. 30, 2015 in the name of ENNAMANY Rachid, both of which are incorporated by reference herein in their entireties. The PCT/IB 2016/000058 application was filed in the name of ENNAMANY Rachid and assigned to NAOLYS SARL as registered at the WIPO on Mar. 3, 2016.

### ABSTRACT OF THE DISCLOSURE

The invention relates to a method for the preparation of in vitro plant cell containing a mixture of molecules in relative proportions between them ensuring an appropriate treatment of problems associated with the skin, meaning being advantageous for cosmetic topical applications.

### THE STATE OF THE ART

Dedifferentiated plant cells means any plant cell exhibiting none of the characters of a particular specialization and capable of living by itself and not in dependence with other cells.

The dedifferentiated plant cells can be obtained from plant material derived from whole plant or plant parts such as leaves, stems, flowers, petals, roots, fruits, skin, shell protecting the seeds, anthers, sap, thorns, shoots, bark, berries, and mixtures thereof.

Preferably, the dedifferentiated plant cells are obtained from bark, leaves, buds and fruit skin.

The dedifferentiated or undifferentiated plant cells used according to the invention may be obtained from plants obtained by culturing in vivo or derived from in vitro culture.

In vivo culture is any conventional culture in soil outdoors or in greenhouses or above ground or in hydroponic medium. By in vitro culture means all the techniques known in the art that can artificially obtain a plant or part of a plant. The selection pressure imposed by the physicochemical conditions during the growth of plant cells in vitro provides a standardized plant material, free from contamination and available throughout the year, unlike the plants cultivated in vivo.

Preferably according to the invention are used dedifferentiated/undifferentiated plant cells from in vitro culture.

The dedifferentiated plant cells used according to the invention may be obtained by any method known to the prior art. In this respect there may be mentioned the methods described by George E. F. and P. D. Sherrington in Plant Propagation by Tissue Culture, Handbook and Directory of Commercial Laboratories (Exegetics Ltd., 1984).

The culture media used in the invention are those generally known to those skilled in the art. One can cite as examples the Gamborg media, Murashige and Skoog, Heller, White, etc . . . . In “Plant Culture Media: formulations and uses”, E. F. George, D J M Puttock and H. J. George (1987, Exegetics Ltd., Volume 1 & 2), complete descriptions of these media can be found.

Preferably according to the invention the dedifferentiated plant cells are cultured on Murashige and Skoog medium.

### PRIOR ART DOCUMENTS

Document FR 2795637 discloses a cosmetic composition containing an extract of dedifferentiated plant cells to avoid odour problems. This composition contains an extract of dedifferentiated plant cells which are not elicited, so that this composition is poor in secondary metabolites or phytoalexins or is substantially free of such compounds. Moreover, this document describes the use of aqueous extract obtained after grinding of the cells in their culture medium and removal of suspended particles with an inevitable loss of metabolites related to suspended particles. To remove proteases and in particular oxidases, this document also advocates the use of filters capturing the molecules with a molecular weight greater than 100,000 daltons, whereby losing in the final extract all the metabolites with molecular weight higher than this weight and which can be of great interest to the cosmetics industry. Further more to eliminate problems due to oxidation, the document recommends the addition of stabilizers, especially cysteine and/or sulfur derivatives which necessarily leads to a lesser purity of the extract with subsequent filtration steps. The methods disclosed in this document may require the implementation of complicated means for obtaining extracts of which both the purity (many additives), the quality and the concentration (in metabolites) are not optimal. Also the many steps necessary for obtaining extracts from this process induce high costs and contamination risk from the many manipulations and additives.

The cultures of dedifferentiated cell cultures are known. On the other hand, the man skilled in the art knows mechanisms of elicitation of these cells followed by steps of extractions and various filtration steps, followed by freeze drying, to incorporate the so prepared extracts in cosmetic or pharmaceutical preparation. Such methods are for example described in U.S. Pat. No. 4,241,536; EP 378 921, WO 88/00968, EP 1203811, etc. for species of various plants. The content of these documents is incorporated herein by reference to describe culture media of plant species, possible elicitors, etc.

Fabiana Antognoni et al (“Induction of flavanoid production by UV-B radiation in Passiflora quadrangularis callus cultures”, 2007, Fitoterapia 78, 345-352) discloses the production of specific flavanoids by exposing culture of Passiflora to a 7-day exposure to UV-B light (280-315 nm) for producing specific flavanoids, namely isoorientin, orientin, isovitexin and vitexin. The test of antioxidant activity shows that after 2 days, the non elicited Passoflora cell extract and the UV elicited Passoflora cell extract had substantially the same antioxidant activity after 2 days, meaning a substantially equivalent activity after 1 day. The elicitation of the Passoflora cell with UVB does not give the appropriate metabolites production for achieving a correct daily antioxidant activity.

From “Plant in vitro culture for the production of antioxidants—A review”, Adam Matkowsky, 2008, Biotechnology Advances 26, 548-560, it is disclosed that chemical classes of antioxidant secondary metabolites can be prepared from plant in vitro cultures. Reference is made in said article to the use of several elicitation means, like bacterial or fungal lysates, stress response mediators (such as salicylate), biotic elicitors, yeast extracts, metal based elicitors, UVB irradiation. No reference at all is made to the elicitation pattern of the invention.

From “Steroid substances in the Yucca gloriosa L. cell and tissue culture and their formation during morphogenesis”, Gogoberidze Mzeinar et al, 1992, Plant science 84, 201-207, extract of intact plant and extract of cell culture of Yucca gloriosa were compared. The document discloses that a drastic change in the proportion of individual sterols in comparison to those of intact plants was observed, and that the processes of differentiation and metabolism had to be further deeper investigated for achieving correct regulation of biosynthesis of secondary substances in plant cell in vitro.

U.S. Pat. No. 4,241,536 claims a process for inducing tumor in vitro comprising treating a growing point of a seedling with a first hormone exhibiting auxin activity and thereafter treating the same growing point of the seedling with a second different hormone exhibiting auxin activity. In particular, it claims a process of embryogenesis in vitro comprising inducing tumor formation in a plant to produce mitotic cells, rejuvenating the mitotic cells in vitro to produce embryonic cells by means of a controlled environment wherein the temperature range is from about 12° to 27° C., the light energy level is from about 2,000 to 400,000 ergs per sq. cm. per second, the spectral range is up to about 730 nm, for a selected photoperiod, and in a suitable nutrient medium, differentiating the embryonic cells to obtain therefrom embryos.

WO 2013/033365 discloses the preparation of aloe vera stem cell in vitro culture under a 16 hour photoperiod with a light intensity of 2000-2500 lux, before being collected and submitted to extraction.

JP2002/193820 relates to skin care preparation containing an Agave extract, and an extra UV light absorbent.

as analysed

Today, despite the skills and expertise of industries in the field of plant extraction, and despite advances in organic chemistry, several extraction steps are necessary to obtain a plant raw material.

Several disadvantages are charged to these extraction steps:
Loss of the tertiary structure of isolated molecules,
The presence of various solvents in the finished product,
Heterogeneity of substrates requiring fine extraction steps requiring more and more toxic solvents,
Quality of the extract depending of the physiological state of the plant at the time of harvest,
Production of the extract limited in function of the seasons.

Given these limiting factors and renewed consumer interest for natural products, several attempts to obtain cells were performed. Thus, to date, two main methods have been commercially operated:
The culture of cells from unicellular organisms or microorganisms, technique based on the reproduction of normal living conditions. However, these organisms are primitive and do not develop secondary metabolism, source of the most interesting active ingredients.
the obtention of cells from fruit (fresh cells) after enzymatic digestion. The limits of this process are that the fruit is not aseptic and may contain residues of pesticides (fungicides, herbicides, insecticides, . . . ). On the other hand, enzymes (cellulases, pectinases, . . . ) used in large quantities (2%, weight/weight) to digest the vegetal cell walls and to obtain cell materials without walls (protoplasts) are present in the final product. Furthermore, the enzymes used can affect the quality of the metabolites. Finally, the use of this technique only allows to recover the protoplasts (cells without cell walls), fragile structures which can not direct their metabolism.

It is also known from document WO 03/077880 a method of preparing a comminuted material of elicited dedifferentiated cells for producing phytoalexins. In said document, the plant cells are selected in particular families and are elicited by specific steps.

The inventor has now discovered that by subjecting dedifferentiated cells of particular plant families to a particular elicitation cycle, it was possible to obtain cells containing a mixture of molecules in relative proportions between them ensuring an appropriate treatment of problems associated with the skin.

### BRIEF DESCRIPTION OF THE INVENTION

The invention relates to a process for the preparation of dedifferentiated and elicited plant cells suitable for topical cosmetic composition, in which dedifferentiated plant cells are put into an in vitro culture medium so as to allow growth of the plant cells, while being submitted to a specific lighting elicitation, not disclosed, nor suggested in the prior art enabling to obtain plant cells containing a mixture of molecules (especially phytoalexins) in appropriate relative proportion the one with respect to the other, ensuring that the said mixture of molecules or phytoalexins is appropriate for treating problems associated with the skin, or for preventing such problems.

In the process of the invention, said lighting elicitation of the growing plant cells in said in vitro culture medium is operated at a temperature comprised between 15° C. and 50° C. under a gazeous atmosphere comprising nitrogen, from 10 to 19% by volume oxygen, from 1 to 10% by volume CO₂ and water (as vapour or gazeous phase) for achieving a relative humidity higher than 50%, whereby said lighting elicitation consists of at least 10 successive darkness periods with a brightness of less than 10 lux separated the one from the other by a lighting period with a brightness of more than 1,000 lux. The nitrogen content (as N₂) in the gas atmosphere without water vapor is advantageously greater than 70% by volume.

The gazeous atmosphere is preferably such that about 99% by volume, preferably more than 99% by volume of the atmosphere consists of N₂, CO₂, O₂ and water vapor.

Each darkness period has a time duration from 20 minutes to 3 hours, while the lighting period between two successive darkness period has a time period from 1 hour to 6 hours, whereby the lighting period has a brightness with substantially no ray with a wavelength of less than 100 nm and of higher than 700 nm, more than 95% of the rays having a wavelength comprised between 400 nm and 520 nm,
while the passage from a darkness period to a lighting period, as well from a lighting period to a darkness period being operated in less than 5 minutes.

In the present specification, when stating that more than 95% or 99% of the rays have a wavelength comprised between 400 nm and 520 nm, it means that more than 95% or 99% of the illuminance (expressed in lux) is due to rays with a wavelength comprised between 400 nm and 520 nm.

The process of the invention has one or more of the following details, advantageously a combination of said following details:
the lighting period has a brightness with substantially no ray with a wavelength of less than 100 nm and of higher than 700 nm, more than 99% of the rays having a wavelength comprised between 400 nm and 520 nm. And/or
the lighting elicitation is operated at a first temperature for the darkness periods and at a second temperature for the lighting periods separating two successive darkness period, said second temperature being higher than the first temperature. And/or
the first temperature is comprised between 15 and 30° C., while the second temperature is comprised between 35 and 50° C. and/or
said lighting elicitation consists of at least ten successive darkness periods with a brightness of less than 10 lux separated the one from the other by a lighting period with a brightness of more than 100,000 lux. And/or
the lighting elicitaion is carried out under a humid atmosphere with a relative humidity higher than 75%, preferably higher than 90%, most preferably at about saturation. And/or
the pressure of the atmosphere is advantageously about the atmospheric pressure or a pressure slightly above the atmospheric pressure, such as a pressure comprised between 0.95×10⁵ Pa and 1.2×10⁵ Pa. and/or
the lighting elicitation is carried out under an humic atmosphere with a relative humidity near to the saturation. And/or
the passage from a darkness period to a lighting period, as well the passage from a lighting period to a darkness period are operated in less than 2 minutes. And/or
the passage from a darkness period to a lighting period, as well the passage from a lighting period to a darkness period are operated in less than 30 seconds, most preferably less than 15 seconds. And/or
the process comprises from 10 to 200 successive darkness periods with a brightness of less than 10 lux separated the one from the other by a lighting period with a brightness of more than 1,000 lux. And/or
the process comprises from 100 to 200 (such as 120, 150, 175, 185) successive darkness periods with a brightness of less than 10 lux separated the one from the other by a lighting period with a brightness of more than 1,000 lux. And/or
The lighting elicitation of the growing plant cells in said in vitro culture medium is operated at a temperature comprised between 15° C. and 50° C. (advantageously between 30 and 50° C.) under a gazeous atmosphere comprising nitrogen, from 10 to 18% (such as 12, 15 17 and 18%) by volume oxygen, from 2 to 7% (such as 2, 3, 5, 6%) by volume CO₂ and water for achieving a relative humidity higher than 75% (advantageously greater than 85%, preferably at about saturation). And/or
The lighting elicitation of the growing plant cells in said in vitro culture medium is operated at a temperature comprised between 15° C. and 50° C. under a gazeous atmosphere comprising nitrogen, from 10 to 18% by volume oxygen, about 5% by volume CO₂ and water for achieving a relative humidity equal to or higher than 75% (advantageously greater than 85%, preferably at about saturation). And/or
the plant cells are selected from the group consisting of the following families: Agavaceae, Aizoaceae, Amaranthaceae, Amaryllidaceae, Anacardiaceae, Apiaceae, Apocynaceae, Araceae; Araliaceae, Asclepiadaceae, Asparagaceae, Asphodelaceae, Asteraceae, Balsaminaceae, Basellaceae, Begoniaceae, Bombacaceae, Brassicaceae, Bromeliaceae, Burseraceae, Cactaceae, Campanulaceae, Capparidaceae, Caricaceae, Chenopodiaceae, Cochlospermaceae, Commelinaceae, Convolvulaceae, Crassulaceae, Cucurbitaceae, Didiereaceae, Dioscoreaceae, Doryanthaceae, Ericaceae, Eriospermaceae, Euphorbiaceae, Fabaceae, Fouquieriaceae, Geraniaceae, Gesneriaceae, Hyacinthaceae, Icacinaceae, Lamiaceae, Lentibulariaceae, Loasaceae, Loranthaceae, Melastomataceae, Meliaceae, Menispermaceae, Moraceae, Moringaceae, Nolanaceae, Nolinaceae, Orchidaceae, Oxalidaceae, Passifloraceae, Pedaliaceae, Phyllanthaceae, Phytolaccaceae, Piperaceae, Portulacaceae, Rubiaceae, Ruscaceae, Sapindaceae, Saxifragaceae, Sterculiaceae, Urticaceae, Viscaceae, Vitaceae, Xanthorrhoeaceae and Zygophyllaceae. And/or
the dedifferentiated and elicited plant cells are submitted after the lighting elicitation to at least one further step selected from the group consisting of: separtion step of plant cells from the culture medium; washing step, drying step, communitation step, mixing step with at least one cosmetic excipient (such as an oil, a glycol, glycerol, etc.), and combinations thereof.

The invention relates also to a process for the preparation of a cosmetic composition for topical application, in which dedifferentiated and elicited plant cells are mixed with at least one cosmetic acceptable excipient, whereby the said dedifferentiated and elicited plant cells are dedifferentiated plant cells which have been elicited into an in vitro culture medium so as to allow growth of the plant cells, while being submitted to a lighting elicitation,
whereby said lighting elicitation of the growing plant cells in said in vitro culture medium is operated at a temperature comprised between 15° C. and 50° C. under a gazeous atmosphere comprising nitrogen, from 10 to 19% by volume oxygen, from 1 to 10% by volume CO₂ and water for achieving a relative humidity higher than 50%, whereby said lighting elicitation consists of at least 10 successive darkness periods with a brightness of less than 10 lux separated the one from the other by a lighting period with a brightness of more than 1,000 lux,
whereby each darkness period has a time duration from 20 minutes to 3 hours, while the lighting period between two successive darkness period has a time period from 1 hour to 6 hours, whereby the lighting period has a brightness with substantially no ray with a wavelength of less than 100 nm and of higher than 700 nm, more than 95% of the rays having a wavelength comprised between 400 nm and 520 nm,
whereby the passage from a darkness period to a lighting period, as well from a lighting period to a darkness period being operated in less than 5 minutes.

The said process has advantageously one or more of the details disclosed herebefore for the preparation of dedifferentiated and elicited plant cells suitable for topical cosmetic composition.

The invention relates also to cosmetic composition for topical application prepared by a process of the invention or containing plant cell prepared by a process according to the invention.

The said composition for topical application of the invention, in particular cosmetic composition, contains plant cells dedifferentiated and elicited in in vitro culture medium, or advantageously ground material of said dedifferentiated plant cells, said at least one phytoalexin containing ground material then comprising at least 95%, advantageously at least 97%, preferably at least 99% by weight of all dry materials from the ground plant cells dedifferentiated and elicited in vitro, said dedifferentiated and elicited plant cells or said ground material being dispersed (s) in said composition or being in a form capable of being dispersed in said composition, characterised in that the dedifferentiated plant cells, optionally in the form of ground material, are selected from the group consisting of the following families: Agavaceae (especially the species: Agave, Beschorneria, Chlorophytum, Furcraea, Hesperaloe, Hesperoyucca, Yucca), Aizoaceae, Amaranthaceae (especially the species: Arthraerva), Amaryllidaceae (especially the species: Boophane, Brunsvigia, Cyrtanthus, Haemanthus, Rauhia), Anacardiaceae (especially the species: Operculicarya, Pachycormus), Apiaceae (especially the species: Steganotaenia), Apocynaceae (especially the species: Adenium, Mandevilla, Pachypodium, Plumeria), Araceae (especially the species: Zamioculcas zamiifolia); Araliaceae (especially the species: Cussonia), Asclepiadaceae (especially the species: Absolmsia, Asclepias, Aspidoglossum, Aspidonepsis, Baynesia, Brachystelma, Caralluma, Ceropegia, Cibirhiza, Cynanchum, Dischidia, Dischidiopsis, Duvalia, Duvaliandra, Echidnopsis, Edithcolea, Fanninia, Fockea, Glossostelma, Hoodia, Hoya, Huernia, Huerniopsis, Ischnolepis, Larryleachia, Lavrania, Madangia, Marsdenia, Matelea, Micholitzia, Miraglossum, Notechidnopsis, Odontostelma, Ophionella, Orbea, Orbeanthus, Pachycarpus, Pectinaria, Petopentia, Piaranthus, Pseudolithos, Quaqua, Raphionacme, Rhytidocaulon, Riocreuxia, Sarcorrhiza, Sarcostemma, Schizoglossum, Schlechterella, Stapelia, Stapelianthus, Stapeliopsis, Stathmostelma, Stenostelma, Stomatostemma, Tavaresia, Trachycalymma, Tridentea, Tromotriche, White-sloanea, Xysmalobium), Asparagaceae (especially the species: Myrsiphyllum), Asphodelaceae (especially the species: Aloe, Astroloba, Bulbine, Chortolirion, Gasteria, Haworthia, Poellnitzia, Trachyandra), Asteraceae (especially the species: Baeriopsis, Coulterella, Crassocephalum, Didelta, Gynura, Osteospermum, Othonna, Polyachyrus, Pteronia, Senecio), Balsaminaceae (especially the species: Impatiens), Basellaceae (especially the species: Anredera, Basella), Begoniaceae (especially the species: Begonia), Bombacaceae (especially the species: Adansonia, Cavanillesia, Ceiba, Pseudobombax), Brassicaceae (especially the species: Heliophila, Lepidium), Bromeliaceae, Burseraceae (especially the species: Beiselia, Bursea, Commiphora), Cactaceae (especially the species: Acanthocalycium, Acanthocereus, Ariocarpus, Armatocereus, Arrojadoa, Arthrocereus, Astrophytum, Austrocactus, Aztekium, Bergerocactus, Blossfeldia, Brachycereus, Browningia, Brasilicereus, Calymmanthium, Carnegiea, Cephalocereus, Cephalocleistocactus, Cereus, Cintia, Cipocereus, Cleistocactus, Coleocephalocereus, Copiapoa, Corryocactus, Coryphantha, Dendrocereus, Denmoza, Discocactus, Disocactus, Echinocactus, Echinocereus, Echinopsis, Epiphyllum, Epithelantha, Eriosyce, Escobaria, Escontria, Espostoa, Espostoopsis, Eulychnia, Facheiroa, Ferocactus, Frailea, Geohintonia, Gymnocalycium, Haageocereus, Harrisia, Hatiora, Hylocereus, Jasminocereus, Lasiocereus, Leocereus, Lepismium, Leptocereus, Leuchtenbergia, Lophophora, Maihuenia, Malacocarpus, Mammillaria, Mammilloydia, Matucana, Melocactus, Micranthocereus, Mila, Monvillea, Myrtillocactus, Neobuxbaumia, Neolloydia, Neoraimondia, Neowerdermannia, Obregonia, Opuntia, Oreocereus, Oroya, Ortegocactus, Pachycereus, Parodia, Pediocactus, Pelecyphora, Peniocereus, Pereskia, Pereskiopsis, Pilosocereus, Polaskia, Praecereus, Pseudoacanthocereus, Pseudorhipsalis, Pterocactus, Pygmaeocereus, Quiabentia, Rauhocereus, Rebutia, Rhipsalis, Samaipaticereus, Schlumbergera, Sclerocactus, Selenicereus, Stenocactus, Stenocereus, Stephanocereus, Stetsonia, Strombocactus, Tacinga, Thelocactus, Turbinicarpus, Uebelmannia, Weberbauerocereus, Weberocereus, Yungasocereus), Campanulaceae (especially the species: Brighamia), Capparidaceae (especially the species: Maerua), Caricaceae (especially the species: Carica, Jacarathia), Chenopodiaceae, Cochlospermaceae, Commelinaceae (especially the species: Aneilema, Callisia, Cyanotis, Tradescantia, Tripogandra), Convolvulaceae (especially the species: Ipomea, Sictocardia, Turbina), Crassulaceae (especially the species: Adromischus, Aeonium, Afrovivella, Aichryson, Cotyledon, Crassula, Cremnophila, Cremnosedum, Dudleya, Echeveria, Graptopetalum, Hylotelephium, Hypagophytum, Kalanchoe, Lenophyllum, Meterostachys, Monanthes, Orostachys, Pachyphytum, Perrierosedum, Phedimus, Pistorinia, Prometheum, Pseudosedum, Rhodiola, Rosularia, Sedella, Sedum, Sempervivum, Sinocrassula, Thompsonella, Tylecodon, Umbilicus, Villadia), Cucurbitaceae (especially the species: Apodanthera, Brandegea, Cephalopentandra, Ceratosanthes, Citrullus, Coccinia, Corallocarpus, Cucumella, Cucumis, Cucurbita, Cyclantheropsis, Dendrosicyos, Doyera, Eureindra, Fevillea, Gerrandanthus, Gynostemma, Halosicyos, Ibervilla, Kedostris, Marah, Momordica, Neoalsomitra, Odosicyos, Parasicyos, Syrigia, Telfairia, Trochomeria, Trochomeriopsis, Tumamoca, Xerosicyos, Zehneria, Zygosicyos), Didiereaceae (especially the species: Alluaudia, Alluaudiopsis, Decaria, Didieara), Dioscoreaceae (especially the species: Dioscorea), Doryanthaceae (especially the species: Doryanthes), Ericaceae (especially the species: Sphyrospermum), Eriospermaceae (especially the species: Eriospermum), Euphorbiaceae, Fabaceae (especially the species: Delonix, Dolichos, Erythrina, Neorautanenia, Pachyrhizus, Tylosema), Fouquieriaceae (especially the species: Fouquieria), Geraniaceae (especially the species: Monsonia, Pelargonium), Gesneriaceae (especially the species: Aeschynanthus, Alsobia, Chirita, Codonanthe, Columnea, Nematanthus, Sinningia, Streptocarpus), Hyacinthaceae (especially the species: Albuca, Bowiea, Dipcadi, Drimia, Drimiopsis, Hyacinthus, Lachenalia, Ledebouria, Litanthus, Massonia, Ornithogalum, Rhadamanthus, Rhodocodon, Schizobasis, Whiteheadia), Icacinaceae (especially the species: Pyrenacantha), Lamiaceae (especially the species: Aeollanthus, Dauphinea, Perrierastrum, Plectranthus, Solenostemon, Tetradenia, Thorncroftia), Lentibulariaceae, Loasaceae (especially the species: Schismocarpus), Loranthaceae (especially the species: Tapinanthus), Melastomataceae (especially the species: Medinilla), Meliaceae (especially the species: Entandrophragma), Menispermaceae (especially the species: Chasmanthera, Stephania, Tinospora), Moraceae (especially the species: Dorstenia, Ficus), Moringaceae (especially the species: Moringa), Nolanaceae (especially the species: Nolana), Nolinaceae (especially the species: Beaucarnea, Calibanus, Dasylirion, Nolina), Orchidaceae, Oxalidaceae (especially the species: Oxalis), Passifloraceae (especially the species: Adenia), Pedaliaceae (especially the species: Pterodiscus, Sesamothamnus, Uncarina), Phyllanthaceae (especially the species: Phyllanthus), Phytolaccaceae (especially the species: Phytolacca), Piperaceae (especially the species: Peperomia), Portulacaceae (especially the species: Amphipetalum, Anacampseros, Avonia, Calyptrotheca, Ceraria, Cistanthe, Dendroportulaca, Grahamia, Lewisia, Parakeelya, Portulaca, Portulacaria, Schreiteria, Talinella, Talinum), Rubiaceae (especially the species: Anthorrhiza, Hydnophytum, Hydrophylax, Myrmecodia, Myrmephythum, Phylohydrax, Squamellaria), Ruscaceae (especially the species: Cordyline, Dracaena, Sansevieria), Sapindaceae (especially the species: Erythrophysa), Saxifragaceae, Sterculiaceae (especially the species: Brachychiton, Sterculia), Urticaceae (especially the species: Laportea, Obertia, Pilea, Sarcopilea), Viscaceae (especially the species: Viscum), Vitaceae (especially the species: Cissus, Cyphostemma), Xanthorrhoeaceae et Zygophyllaceae, which have been elicited as stated in the process of the invention.

In the present specification, the periods of non brightness are advantageously periods for which the culture medium is substantially not subjected to a radiation with a wavelength comprised between 100 nm and 700 nm, but also preferably with wavelength of less than 100 nm and of more than 700 nm. Periods of non brightness are preferably periods of total or substantially total darkness.

According to an advantageous embodiment of the composition according to the invention, the elicited and dedifferentiated plant cells are subjected to a removal step from the culture medium and/or a washing step and/or a rinsing step and/or a drying step. The said cells, preferably after separation from the culture medium and/or washing and/or drying, are preferably ground before or after mixing them to a compound acceptable in cosmetics.

The cells are possibly, advantageously in the form of a ground or cominuted material of cells dedifferentiated and elicited in an in vitro medium, said cells being at least partially dried, preferably lyophilized, prior to grinding.

The composition contains from 0.005 to 25% by weight, preferably from 0.005 to 5% by weight of plant cells dedifferentiated and elicited in an in vitro medium, in particular in the form of ground material, this weight being calculated in dry form.

The ground material the ground material has an average particle size of solid particles of less than 100 μm, advantageously less than 10 μm, preferably less than 1 μm.

The unground plant cells dedifferentiated and elicited in an in vitro medium or ground material of plant cells dedifferentiated and elicited in an in vitro medium is (are) in the form of a viscous suspension or a gel or a substantially dry powder, said suspension, gel or powder being advantageously in a form suitable for being dispersed in the composition.

The composition of the invention can be an antioxidant composition, anti-free radical composition, anti inflammatory composition, anti-proliferative composition, relaxing composition, vascular composition and/or anti-aging composition, said composition comprising an effective amount of plant cells as described above and/or plant cell ground material as described above, prepared by a process of the invention.

The cells are advantageously subjected to a grinding step, advantageously after a washing step of the cells and/or a drying step of the cells and/or a mixing step of the cells with one or more excipients for cosmetic application.

Said cells elicited in an in vitro medium are submitted to a drying, advantageously a lyophilization, optionally followed by grinding, before mixing the cells or ground material with one or more acceptable excipients for topical application.

The dedifferentiated plant cells are cultured in an in vitro culture medium, are elicited in the in vitro culture medium, dried, and optionally ground, optionally after one or more washing and/or rinsing and/or drying steps, and dispersed in the human body treatment composition.

The advantage of the processes of the invention is that it provides plant cells rich in particular molecules (of the family from the stilbene, flavanoids, néobëtanine, alkaloids, vitamins, quercetin 3-methyl ether, fatty acid derivatives of rutin or rutinoside, Gallic acid, isorhamnetin, etc.) in adequate proportions in large volumes, while meeting the needs of the industry, including:
Compliance with the tertiary structure of the molecules,
The absence of solvent and residues,
The homogeneity of substrates,
Production continues regardless of the cycle of the seasons,
The conservation of biological and physiological characteristics without addition of preservative,
The total absence of pollutants,
The standardized and reproducible production with the quality and concentration of metabolites,
The use of these elicited dedifferentiated plant cell suspension after lyophilization at temperature of less than −30° C. This technique allows the obtention of a very fine powder (containing the whole content of the plant cells) that can be dispersed in cosmetic compositions (creams, ointments, lotions . . . ).
The fresh elicited dedifferentiated plant cells are capable of releasing the active ingredients they contain directly when applied on the skin, without passing through an extraction step using organic solvents (whereby elimining the risks of solvent residues).
The use of fresh elicited dedifferentiated plant cell content, prepared after sonication and centrifugation.
The use of fresh undifferentiated elicited plant cells, not ground, mixed with one or more cosmetic excipients (eg glycerin, glycol (s), oil (s), etc.). Fresh cells are advantageously isolated from the culture medium, washed and optionally rinsed prior to being optionally dried, before being mixed with one or more excipients for cosmetic application.

This technology provides a useful and innovative alternative to conventional solvent extractions. The ability to orient naturally (elicitation) synthesis of metabolites without affecting the genetic integrity of cells represents a guarantee of quality and authenticity.

So quite surprisingly the inventor has discovered that the cells after specific elicitation, possibly after further drying and/or grinding, could directly be incorporated or dispersed in a cosmetic and/or pharmaceutical composition. The composition according to the invention then contains all the material of the fresh elicited dedifferentiated plant cells, including the plant cell membranes. This method has the advantage to ensure a specific elicitation without adding liquid/solid additives or chemicals, which are not present in the culture medium. Another aspect of the invention allows focusing and directing the production of phytoalexins without qualitative or quantitative losses due to extraction and filtering. A particular aspect of the invention is that it avoids the steps of extraction and filtration and allows obtaining a ground material of cells devoid of additives, solvents and residues, said ground material can directly be dispersed in a cosmetic composition.

Composition for topical application means: creams, ointments, lotions, suspensions, sticks, shampoos, gels, serums, milks, lotions, creams, solutions (eg applied by spray). The topical composition is for example a cosmetic, dermatological, a skin hygiene composition, a perfume, etc.

Preferably according to the invention, the composition is a cosmetic composition.

The following examples and compositions illustrate the invention without limiting it in any way. In the compositions, indicated proportions are percentages by weight.

In these examples, a preferred process as defined below was used.

### A Method of Obtaining Fresch Elicited Dedifferentiated Plant Cell or a Ground Material Thereof.

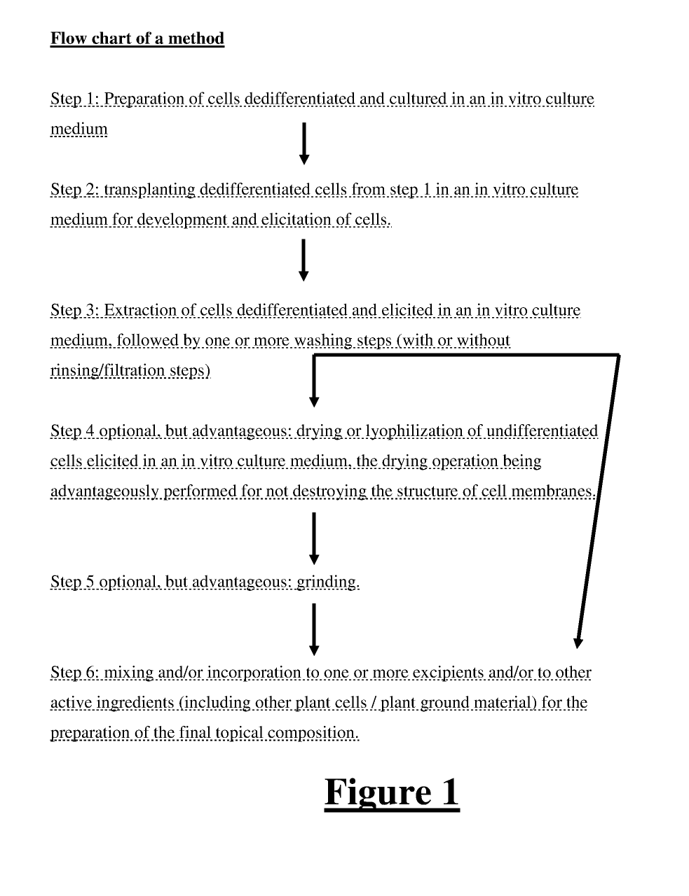

Reference is made to FIG. 1 giving the general flow chart of the said method.

### Step 1: Preparation of Cells Dedifferentiated and Cultured in an In Vitro Culture Medium

This preparation step of dedifferentiated cells was performed conventionally. For this step, plant cells from plants of the following families were used:

Agavaceae (especially the species: Agave, Beschorneria, Chlorophytum, Furcraea, Hesperaloe, Hesperoyucca, Yucca), Aizoaceae, Amaranthaceae (especially the species: Arthraerva), Amaryllidaceae (especially the species: Boophane, Brunsvigia, Cyrtanthus, Haemanthus, Rauhia), Anacardiaceae (especially the species: Operculicarya, Pachycormus), Apiaceae (especially the species: Steganotaenia), Apocynaceae (especially the species: Adenium, Mandevilla, Pachypodium, Plumeria), Araceae (especially the species: Zamioculcas zamiifolia); Araliaceae (especially the species: Cussonia), Asclepiadaceae (especially the species: Absolmsia, Asclepias, Aspidoglossum, Aspidonepsis, Baynesia, Brachystelma, Caralluma, Ceropegia, Cibirhiza, Cynanchum, Dischidia, Dischidiopsis, Duvalia, Duvaliandra, Echidnopsis, Edithcolea, Fanninia, Fockea, Glossostelma, Hoodia, Hoya, Huernia, Huerniopsis, Ischnolepis, Larryleachia, Lavrania, Madangia, Marsdenia, Matelea, Micholitzia, Miraglossum, Notechidnopsis, Odontostelma, Ophionella, Orbea, Orbeanthus, Pachycarpus, Pectinaria, Petopentia, Piaranthus, Pseudolithos, Quaqua, Raphionacme, Rhytidocaulon, Riocreuxia, Sarcorrhiza, Sarcostemma, Schizoglossum, Schlechterella, Stapelia, Stapelianthus, Stapeliopsis, Stathmostelma, Stenostelma, Stomatostemma, Tavaresia, Trachycalymma, Tridentea, Tromotriche, White-sloanea, Xysmalobium), Asparagaceae (especially the species: Myrsiphyllum), Asphodelaceae (especially the species: Aloe, Astroloba, Bulbine, Chortolirion, Gasteria, Haworthia, Poellnitzia, Trachyandra), Asteraceae (especially the species: Baeriopsis, Coulterella, Crassocephalum, Didelta, Gynura, Osteospermum, Othonna, Polyachyrus, Pteronia, Senecio), Balsaminaceae (especially the species: Impatiens), Basellaceae (especially the species: Anredera, Basella), Begoniaceae (especially the species: Begonia), Bombacaceae (especially the species: Adansonia, Cavanillesia, Ceiba, Pseudobombax), Brassicaceae (especially the species: Heliophila, Lepidium), Bromeliaceae, Burseraceae (especially the species: Beiselia, Bursea, Commiphora), Cactaceae (especially the species: Acanthocalycium, Acanthocereus, Ariocarpus, Armatocereus, Arrojadoa, Arthrocereus, Astrophytum, Austrocactus, Aztekium, Bergerocactus, Blossfeldia, Brachycereus, Browningia, Brasilicereus, Calymmanthium, Carnegiea, Cephalocereus, Cephalocleistocactus, Cereus, Cintia, Cipocereus, Cleistocactus, Coleocephalocereus, Copiapoa, Corryocactus, Coryphantha, Dendrocereus, Denmoza, Discocactus, Disocactus, Echinocactus, Echinocereus, Echinopsis, Epiphyllum, Epithelantha, Eriosyce, Escobaria, Escontria, Espostoa, Espostoopsis, Eulychnia, Facheiroa, Ferocactus, Frailea, Geohintonia, Gymnocalycium, Haageocereus, Harrisia, Hatiora, Hylocereus, Jasminocereus, Lasiocereus, Leocereus, Lepismium, Leptocereus, Leuchtenbergia, Lophophora, Maihuenia, Malacocarpus, Mammillaria, Mammilloydia, Matucana, Melocactus, Micranthocereus, Mila, Monvillea, Myrtillocactus, Neobuxbaumia, Neolloydia, Neoraimondia, Neowerdermannia, Obregonia, Opuntia, Oreocereus, Oroya, Ortegocactus, Pachycereus, Parodia, Pediocactus, Pelecyphora, Peniocereus, Pereskia, Pereskiopsis, Pilosocereus, Polaskia, Praecereus, Pseudoacanthocereus, Pseudorhipsalis, Pterocactus, Pygmaeocereus, Quiabentia, Rauhocereus, Rebutia, Rhipsalis, Samaipaticereus, Schlumbergera, Sclerocactus, Selenicereus, Stenocactus, Stenocereus, Stephanocereus, Stetsonia, Strombocactus, Tacinga, Thelocactus, Turbinicarpus, Uebelmannia, Weberbauerocereus, Weberocereus, Yungasocereus), Campanulaceae (especially the species: Brighamia), Capparidaceae (especially the species: Maerua), Caricaceae (especially the species: Carica, Jacarathia), Chenopodiaceae, Cochlospermaceae, Commelinaceae (especially the species: Aneilema, Callisia, Cyanotis, Tradescantia, Tripogandra), Convolvulaceae (especially the species: Ipomea, Sictocardia, Turbina), Crassulaceae (especially the species: Adromischus, Aeonium, Afrovivella, Aichryson, Cotyledon, Crassula, Cremnophila, Cremnosedum, Dudleya, Echeveria, Graptopetalum, Hylotelephium, Hypagophytum, Kalanchoe, Lenophyllum, Meterostachys, Monanthes, Orostachys, Pachyphytum, Perrierosedum, Phedimus, Pistorinia, Prometheum, Pseudosedum, Rhodiola, Rosularia, Sedella, Sedum, Sempervivum, Sinocrassula, Thompsonella, Tylecodon, Umbilicus, Villadia), Cucurbitaceae (especially the species: Apodanthera, Brandegea, Cephalopentandra, Ceratosanthes, Citrullus, Coccinia, Corallocarpus, Cucumella, Cucumis, Cucurbita, Cyclantheropsis, Dendrosicyos, Doyera, Eureindra, Fevillea, Gerrandanthus, Gynostemma, Halosicyos, Ibervilla, Kedostris, Marah, Momordica, Neoalsomitra, Odosicyos, Parasicyos, Syrigia, Telfairia, Trochomeria, Trochomeriopsis, Tumamoca, Xerosicyos, Zehneria, Zygosicyos), Didiereaceae (especially the species: Alluaudia, Alluaudiopsis, Decaria, Didieara), Dioscoreaceae (especially the species: Dioscorea), Doryanthaceae (especially the species: Doryanthes), Ericaceae (especially the species: Sphyrospermum), Eriospermaceae (especially the species: Eriospermum), Euphorbiaceae, Fabaceae (especially the species: Delonix, Dolichos, Erythrina, Neorautanenia, Pachyrhizus, Tylosema), Fouquieriaceae (especially the species: Fouquieria), Geraniaceae (especially the species: Monsonia, Pelargonium), Gesneriaceae (especially the species: Aeschynanthus, Alsobia, Chirita, Codonanthe, Columnea, Nematanthus, Sinningia, Streptocarpus), Hyacinthaceae (especially the species: Albuca, Bowiea, Dipcadi, Drimia, Drimiopsis, Hyacinthus, Lachenalia, Ledebouria, Litanthus, Massonia, Ornithogalum, Rhadamanthus, Rhodocodon, Schizobasis, Whiteheadia), Icacinaceae (especially the species: Pyrenacantha), Lamiaceae (especially the species: Aeollanthus, Dauphinea, Perrierastrum, Plectranthus, Solenostemon, Tetradenia, Thorncroftia), Lentibulariaceae, Loasaceae (especially the species: Schismocarpus), Loranthaceae (especially the species: Tapinanthus), Melastomataceae (especially the species: Medinilla), Meliaceae (especially the species: Entandrophragma), Menispermaceae (especially the species: Chasmanthera, Stephania, Tinospora), Moraceae (especially the species: Dorstenia, Ficus), Moringaceae (especially the species: Moringa), Nolanaceae (especially the species: Nolana), Nolinaceae (especially the species: Beaucarnea, Calibanus, Dasylirion, Nolina), Orchidaceae, Oxalidaceae (especially the species: Oxalis), Passifloraceae (especially the species: Adenia), Pedaliaceae (especially the species: Pterodiscus, Sesamothamnus, Uncarina), Phyllanthaceae (especially the species: Phyllanthus), Phytolaccaceae (especially the species: Phytolacca), Piperaceae (especially the species: Peperomia), Portulacaceae (especially the species: Amphipetalum, Anacampseros, Avonia, Calyptrotheca, Ceraria, Cistanthe, Dendroportulaca, Grahamia, Lewisia, Parakeelya, Portulaca, Portulacaria, Schreiteria, Talinella, Talinum), Rubiaceae (especially the species: Anthorrhiza, Hydnophytum, Hydrophylax, Myrmecodia, Myrmephythum, Phylohydrax, Squamellaria), Ruscaceae (especially the species: Cordyline, Dracaena, Sansevieria), Sapindaceae (especially the species: Erythrophysa), Saxifragaceae, Sterculiaceae (especially the species: Brachychiton, Sterculia), Urticaceae (especially the species: Laportea, Obertia, Pilea, Sarcopilea), Viscaceae (especially the species: Viscum), Vitaceae (especially the species: Cissus, Cyphostemma), Xanthorrhoeaceae et Zygophyllaceae

This step 1 is carried out in a clean room under an atmosphere of sterile air (at about atmospheric pressure or just above atmospheric pressure), with a constant lighting or illuminance of more than 100,000 lux, at a temperature of 30° C. and a relative humidity of 50% or more than 50% (such as at about saturation). This step is carried out by successive replanting a portion of the plant, in particular a portion of the root. Lighting was the type emitting more than 95% of rays illuminance (in particular more than 99%) with rays in the range of 100 nm to 700 nm, preferably more than 95%, most preferably more than 99% of the ray illuminance being due to rays within the range of 400-520 nm.

Step 2: Transplanting Dedifferentiated Cells from Step 1 in an In Vitro Culture Medium for Development and Elicitation of Cells.

The development with elicitation of the cells of step 1 was operated in an in vitro medium under a gaseous atmosphere (at a pressure comprised between 0.9×10⁵ Pa and 1.2×10⁵ Pa) containing oxygen and CO₂ according to a cycle comprising 100 periods of low light with a brightness of less than 10 lux (of 1 to 5 lux) for 1 hour under an atmosphere consisting of moist air (relative humidity 75% or higher) enriched with CO₂ (so that the CO₂ content is about 5%) and having a temperature of 30° C., these periods development/elicitation under low (or no) lighting (preferably substantially complete darkness) being separated from each other by a lighting period or lighting of more than 100,000 lux (The lighting was of the type emitting more than 95% of rays (in particular more than 99%) in the range of 100 nm to 700 nm, preferably 400-520 nm) for 1 hour under an atmosphere of humid air (relative humidity 75%) enriched with CO₂ (so that the CO₂ content of the atmosphere or gazeous medium is 5%) and having a temperature of about 45° C. The transition from a darkness state to a lighting state is realized by the movement of an opaque wall. This culture is made in clean or sterile or aseptic room.

It was noted that this culture and elicitation method of cells enables to obtain a higher content of phytoalexins, flavanoids (rutin, gallic acid, derivatives of isorhamnetin, etc.) relative to that obtained by in vitro culture under a gas atmosphere (at a pressure comprised between 0.9×10⁵ Pa and 1.2×10⁵ Pa) containing oxygen and CO₂ according to a constant lighting cycle, but even according to a cycle comprising periods of low brightness with a brightness of less than 10 lux (1 to 5 lux) for 12 to 24 hours under an atmosphere consisting of moist air (relative humidity 75% or higher) enriched ith CO₂ (so that the CO₂ content is about 5%) and having a temperature of 30° C., these periods of low (or no) lighting being separated from each other by a period of brightness of more than 100,000 lux for 12 hours under an atmosphere of humid air (relative humidity 75% or higher) enriched with CO₂ (so that the atmospheric CO₂ content is about 5%) and having a temperature of about 45° C.

It was also noted that the enrichment in CO₂ of the atmosphere, as well as the absence of UVB ray for the elicitation and the darkness period between two lighting or illumination periods had a positive effect on the production of phytoalexins, flavanoids, etc., as well as on the relative proportion of one phytoalexin with respect to another.

Step 3: Extraction of Cells Dedifferentiated and Elicited in an In Vitro Culture Medium, for Example by Filtration of the Culture Medium, Followed by One or More Washing Steps (with or without Rinsing/Filtration), in Particular Operated so as not to Destroy the Structure of the Cell Membranes.

The cells dedifferentiated and elicited in culture medium, after washing, can directly be mixed with one or more cosmetic excipients (see step 6).

For example, fresh cells are mixed with glycerol and/or one or more glycols and/or one or more oils (advantageously plant oils), the weight proportion of the mixture in plant cells being preferably 1 to 20%, said mixture then constituting a product suitable for use in the preparation of a cosmetic composition.

Step 4 Optional, but Advantageous: Drying or Lyophilization of Undifferentiated Cells Elicited in an In Vitro Culture Medium, the Drying Operation being Advantageously. Performed for not Destroying the Structure of Cell Membranes.

This step is advantageously carried out at a temperature below 60° C., for example at a temperature comprised between −60° C. and +50° C.

### Step 5 Optional, but Advantageous: Grinding.

Grinding is operated so that the particle size is less than 50 μm, especially less than 10 μm, such as an weight average particle size of 5 μm or less than 5 μm. It is interesting to perform the grinding operation of elicited dedifferentiated cells in the presence of one or more agents or excipients of the final cosmetic composition to ensure the release of phytoalexins and other compounds from the fresh cells directly in at least one or more agents of the final cosmetic composition.

Step 6: Mixing and/or Incorporation to One or More Excipients and/or to Other Active Ingredients (Including Other Plant Cells/Plant Ground Material) for the Preparation of the Final Topical Composition.

For example, a mixture of not comminuted fresh elicited dedifferentiated plant cells in glycerol and/or one or more glycols and/or one or more oils is used for preparing a composition ready to be used for the preparation of a topical cosmetic composition by addition of one or additional excipients.

When using fresch elicited de differentiated plant cells, said fresh plant cells are removed from the culture medium, washed with water, and filtered for removing the excess of water.

### Example of Antioxidant Pharmacological Activity

The anti-radical activity of cells or plant cell ground material prepared by the method described above was studied in vitro, using a model of reconstituted SKINETHIC® epiderms, enabling determine said activity by dosing the malondialdehyde (MDA), after induction with ultraviolet rays B.

In conclusion, the cells or the ground material prepared by the method of the present invention have an anti-radical activity both in physiological conditions and in the induction conditions by ultraviolet radiation B. It is clear from this test that the ground material has a significant anti-radical effect.

### Example of Elicited Cells Dispersion (Preferably Ground) in a Cosmetic Base

Plant cells prepared as described above are used for the preparation of a cosmetic composition. The cells are dispersed after lyophilization without being ground in the following base composition:

| | |
|---|---|
| demineralized water | 85.61% |
| Mineral oil | 9.00% |
| Cetyl alcohol | 3.00% |
| ceteareth - 20 | 0.75% |
| plant cells (ground or not) | 0.20% |
| Perfume | 0.15% |
| carbomer | 0.10% |
| methylchloroisothiazoline | 0.065% |
| and methylisothiazolin [Kathon CG] | |
| Sodium hydroxide (45%) | 0.06% |
| Butyl hydroxyanisole | 0.06% |
| TOTAL | 100.00% |

The composition obtained shows a homogeneous dispersion of the fresh elicited dedifferentiated plant cells or cell ground materials issued from fresh elicited dedifferentiated plant cells in the cream and a very fine particle size. The safety study has showed the absence of germs and fungi and a remarkable stability of the composition. The resulted composition tested in a transcutaneous assay enables to observe the passage of active ingredients including polyphenols and flavonoids through the skin tissue.

Plant cells (ground or not) may be used in creams, lotions, shampoo, gels, solutions, milks.

The proportion of cells or ground cells, in the form of a viscous suspension or a gel or a substantially dry powder is depending on the nature of the topical composition and desired application. Said weight content is advantageously between 0.01 and 5%, but can reach 25%.

Obviously, the invention is not limited to the embodiments given above and it is possible to make the composition for topical use in other forms, such as oil, ointment, lacquers, cosmetics (foundation, powder, lipstick, pencil, mascara, eye shadow) which are also within the scope of the invention.

Cosmetic compositions according to the invention (with a weight content of 1% ground material of plant cells—in dry form) were tested on volunteers. It has been observed that such compositions had an anti-aging effect, a protective effect for the skin, an antioxidant effect, anti-radical, anti fungal, anti acne, rejuvenation effect of the skin, etc.

Cosmetic compositions comprising unground fresh washed elicited dedifferentiated plant cells were also tested on volunteers. Said compositions had also an anti-aging effect, a protective effect for the skin, an antioxidant effect, anti-radical, anti fungal, anti acne, rejuvenation effect of the skin, etc.

What I claim is:

1. A process for the preparation of dedifferentiated and elicited plant cells suitable for topical cosmetic composition, in which dedifferentiated plant cells are put into an in vitro culture medium so as to allow growth of the plant cells, while being submitted to a lighting elicitation,
whereby said lighting elicitation of the growing plant cells in said in vitro culture medium is operated at a temperature comprised between 15° C. and 50° C. under a gazeous atmosphere comprising nitrogen, from 10 to 19% by volume oxygen, from 1 to 10% by volume CO₂ and water for achieving a relative humidity higher than 50%, whereby said lighting elicitation consists of at least 10 successive darkness periods with a brightness of less than 10 lux separated the one from the other by a lighting period with a brightness of more than 1,000 lux,
whereby each darkness period has a time duration from 20 minutes to 3 hours, while the lighting period between two successive darkness period has a time period from 1 hour to 6 hours, whereby the lighting period has a brightness with substantially no ray with a wavelength of less than 100 nm and of higher than 700 nm, more than 95% of the rays having a wavelength comprised between 400 nm and 520 nm,
whereby the passage from a darkness period to a lighting period, as well from a lighting period to a darkness period being operated in less than 5 minutes.

2. The process of claim 1, in which the lighting period has a brightness with substantially no ray with a wavelength of less than 100 nm and of higher than 700 nm, more than 99% of the rays having a wavelength comprised between 400 nm and 520 nm.

3. The process of claim 1, in which the lighting elicitation is operated at a first temperature for the darkness periods and at a second temperature for the lighting periods separating two successive darkness period, said second temperature being higher than the first temperature.

4. The process of claim 3, in which the first temperature is comprised between 15 and 30° C., while the second temperature is comprised between 35 and 50° C.

5. The process of claim 1, in which said lighting elicitation consists of at least ten successive darkness periods with a brightness of less than 10 lux separated the one from the other by a lighting period with a brightness of more than 100,000 lux.

6. The process of claim 1, in which the lighting elicitation is carried out under a humid atmosphere with a relative humidity higher than 75%.

7. The process of claim 1, in which the lighting elicitation is carried out under an humic atmosphere with a relative humidity near to the saturation.

8. The process of claim 1, in which the passage from a darkness period to a lighting period, as well the passage from a lighting period to a darkness period are operated in less than 2 minutes.

9. The process of claim 1, in which the passage from a darkness period to a lighting period, as well the passage from a lighting period to a darkness period are operated in less than 30 seconds.

10. The process of claim 1, which comprises from 10 to 200 successive darkness periods with a brightness of less than 10 lux separated the one from the other by a lighting period with a brightness of more than 1,000 lux.

11. The process of claim 1, which comprises from 100 to 200 successive darkness periods with a brightness of less than 10 lux separated the one from the other by a lighting period with a brightness of more than 1,000 lux.

12. The process of claim 1, in which said lighting elicitation of the growing plant cells in said in vitro culture medium is operated at a temperature comprised between 15° C. and 50° C. under a gazeous atmosphere comprising nitrogen, from 10 to 18% by volume oxygen, from 2 to 7% by volume CO₂ and water for achieving a relative humidity equal to or higher than 75%.

13. The process of claim 1, in which said lighting elicitation of the growing plant cells in said in vitro culture medium is operated at a temperature comprised between 15° C. and 50° C. under a gazeous atmosphere comprising nitrogen, from 10 to 18% by volume oxygen, about 5% by volume CO₂ and water for achieving a relative humidity higher than 75%.

14. The process of claim 1, in which the plant cells are selected from the group consisting of the following families: Agavaceae, Aizoaceae, Amaranthaceae, Amaryllidaceae, Anacardiaceae, Apiaceae, Apocynaceae, Araceae; Araliaceae, Asclepiadaceae, Asparagaceae, Asphodelaceae, Asteraceae, Balsaminaceae, Basellaceae, Begoniaceae, Bombacaceae, Brassicaceae, Bromeliaceae, Burseraceae, Cactaceae, Campanulaceae, Capparidaceae, Caricaceae, Chenopodiaceae, Cochlospermaceae, Commelinaceae, Convolvulaceae, Crassulaceae, Cucurbitaceae, Didiereaceae, Dioscoreaceae, Doryanthaceae, Ericaceae, Eriospermaceae, Euphorbiaceae, Fabaceae, Fouquieriaceae, Geraniaceae, Gesneriaceae, Hyacinthaceae, Icacinaceae, Lamiaceae, Lentibulariaceae, Loasaceae, Loranthaceae, Melastomataceae, Meliaceae, Menispermaceae, Moraceae, Moringaceae, Nolanaceae, Nolinaceae, Orchidaceae, Oxalidaceae, Passifloraceae, Pedaliaceae, Phyllanthaceae, Phytolaccaceae, Piperaceae, Portulacaceae, Rubiaceae, Ruscaceae, Sapindaceae, Saxifragaceae, Sterculiaceae, Urticaceae, Viscaceae, Vitaceae, Xanthorrhoeaceae and Zygophyllaceae.

15. The process of claim 1, in which the dedifferentiated and elicited plant cells are submitted after the lighting elicitation to at least one further step selected from the group consisting of: separtion step of plant cells from the culture medium; washing step, drying step, communitation step, mixing step with at least one cosmetic excipient, and combinations thereof.

16. A process for the preparation of a cosmetic composition for topical application, in which dedifferentiated and elicited plant cells are mixed with at least one cosmetic acceptable excipient, whereby the said dedifferentiated and elicited plant cells are
dedifferentiated plant cells which have been elicited into an in vitro culture medium so as to allow growth of the plant cells, while being submitted to a lighting elicitation,
whereby said lighting elicitation of the growing plant cells in said in vitro culture medium is operated at a temperature comprised between 15° C. and 50° C. under a gazeous atmosphere comprising nitrogen, from 10 to 19% by volume oxygen, from 1 to 10% by volume CO₂ and water for achieving a relative humidity higher than 50%, whereby said lighting elicitation consists of at least 10 successive darkness periods with a brightness of less than 10 lux separated the one from the other by a lighting period with a brightness of more than 1,000 lux,
whereby each darkness period has a time duration from 20 minutes to 3 hours, while the lighting period between two successive darkness period has a time period from 1 hour to 6 hours, whereby the lighting period has a brightness with substantially no ray with a wavelength of less than 100 nm and of higher than 700 nm, more than 95% of the rays having a wavelength comprised between 400 nm and 520 nm,
whereby the passage from a darkness period to a lighting period, as well from a lighting period to a darkness period being operated in less than 5 minutes.

17. The process of claim 16, in which the lighting period has a brightness with substantially no ray with a wavelength of less than 100 nm and of higher than 700 nm, more than 99% of the rays having a wavelength comprised between 400 nm and 520 nm.

18. The process of claim 16, in which the lighting elicitation is operated at a first temperature for the darkness periods and at a second temperature for the lighting periods separating two successive darkness period, said second temperature being higher than the first temperature.

19. The process of claim 18, in which the first temperature is comprised between and 30° C., while the second temperature is comprised between 35 and 50° C.

20. The process of claim 16, in which said lighting elicitation consists of at least ten successive darkness periods with a brightness of less than 10 lux separated the one from the other by a lighting period with a brightness of more than 100,000 lux.

21. The process of claim 16, in which the lighting elicitation is carried out under a humid atmosphere with a relative humidity higher than 75%.

22. The process of claim 16, in which the lighting elicitation is carried out under an humic atmosphere with a relative humidity near to the saturation.

23. The process of claim 16, in which the passage from a darkness period to a lighting period, as well the passage from a lighting period to a darkness period are operated in less than 2 minutes.

24. The process of claim 16, in which the passage from a darkness period to a lighting period, as well the passage from a lighting period to a darkness period are operated in less than 30 seconds.

25. The process of claim 16, which comprises from 10 to 200 successive darkness periods with a brightness of less than 10 lux separated the one from the other by a lighting period with a brightness of more than 1,000 lux.

26. The process of claim 16, which comprises from 100 to 200 successive darkness periods with a brightness of less than 10 lux separated the one from the other by a lighting period with a brightness of more than 1,000 lux.

27. The process of claim 16, in which said lighting elicitation of the growing plant cells in said in vitro culture medium is operated at a temperature comprised between 15° C. and 50° C. under a gazeous atmosphere comprising nitrogen, from 10 to 18% by volume oxygen, from 2 to 7% by volume CO₂ and water for achieving a relative humidity higher than 75%.

28. The process of claim 16, in which said lighting elicitation of the growing plant cells in said in vitro culture medium is operated at a temperature comprised between 15° C. and 50° C. under a gazeous atmosphere comprising nitrogen, from 10 to 18% by volume oxygen, about 5% by volume CO₂ and water for achieving a relative humidity higher than 75%.

29. The process of claim 16, in which the plant cells are selected from the group consisting of the following families: Agavaceae, Aizoaceae, Amaranthaceae, Amaryllidaceae, Anacardiaceae, Apiaceae, Apocynaceae, Araceae; Araliaceae, Asclepiadaceae, Asparagaceae, Asphodelaceae, Asteraceae, Balsaminaceae, Basellaceae, Begoniaceae, Bombacaceae, Brassicaceae, Bromeliaceae, Burseraceae, Cactaceae, Campanulaceae, Capparidaceae, Caricaceae, Chenopodiaceae, Cochlospermaceae, Commelinaceae, Convolvulaceae, Crassulaceae, Cucurbitaceae, Didiereaceae, Dioscoreaceae, Doryanthaceae, Ericaceae, Eriospermaceae, Euphorbiaceae, Fabaceae, Fouquieriaceae, Geraniaceae, Gesneriaceae, Hyacinthaceae, Icacinaceae, Lamiaceae, Lentibulariaceae, Loasaceae, Loranthaceae, Melastomataceae, Meliaceae, Menispermaceae, Moraceae, Moringaceae, Nolanaceae, Nolinaceae, Orchidaceae, Oxalidaceae, Passifloraceae, Pedaliaceae, Phyllanthaceae, Phytolaccaceae, Piperaceae, Portulacaceae, Rubiaceae, Ruscaceae, Sapindaceae, Saxifragaceae, Sterculiaceae, Urticaceae, Viscaceae, Vitaceae, Xanthorrhoeaceae and Zygophyllaceae.

30. The process of claim 16, in which the dedifferentiated and elicited plant cells are submitted after the lighting elicitation to at least one further step selected from the group consisting of: separtion step of plant cells from the culture medium; washing step, drying step, communitation step, mixing step with at least one cosmetic excipient, and combinations thereof.
